(19) United States
(12) Patent Application Publication
(10) Pub. No.: US 2007/0149794 A1
(43) Pub. Date: Jun. 28, 2007

(54) Process for preparing optically active 3-azidocarboxylic acid derivatives and 3-aminocarboxylic acid derivatives

(75) Inventor: Andreas Bohm, Munchen (DE)

Correspondence Address: BROOKS KUSHMAN P.C., 1000 TOWN CENTER, TWENTY-SECOND FLOOR, SOUTHFIELD, MI, 48075, US

(73) Assignee: Wacker Chemie AG, Munich (DE)

(21) Appl. No.: 11/639,741

(22) Filed: Dec. 15, 2006

(30) Foreign Application Priority Data
Dec. 22, 2005 (DE) 10 2005 061 575.9

(51) Int. Cl. C07C 247/04

(52) U.S. Cl. 552/11; 552/12

(57) ABSTRACT

A process for enantioselectively preparing 3-azidocarboxylic acid derivatives comprises reacting 3-sulfonatocarboxylic acid derivatives with an alkali metal azide in a solvent selected from the group comprising certain carboxamides; a solvent mixture which comprises such carboxamides; a solvent mixture of water and a solvent miscible homogeneously with water; water with the proviso that the addition of a phase transfer catalyst is not used in the reaction in water; and DMSO. The resulting products are optionally reduced to 3-aminocarboxylic acid derivatives.

### BACKGROUND OF THE INVENTION

1. Field of the Invention

The present invention relates to a process for preparing optically active 3-azidocarboxylic acid derivatives of the general formula (III) and optically active 3-aminocarboxylic acid derivatives of the general formula (IV) by reacting sulfonates of the general formula (II) which are based on the optically active 3-hydroxycarboxylic acid derivatives of the general formula (I), wherein the radicals R, R¹, X, and Y are each as defined below.

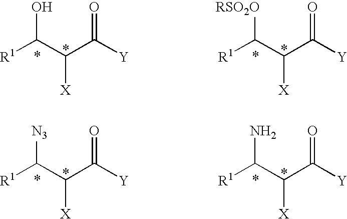

formula (I) formula (II) formula (III) formula (IV)

### The target compounds of the formulae (III) and (IV) can be used in particular as an intermediate in the preparation of active pharmaceutical ingredients and are thus of great industrial and economic interest.

2. Background Art

The stereoselective azidation of optically active 3-hydroxycarboxylic acid derivatives of the general formula (I) via sulfonates of the general formula (II) to give the corresponding optically active 3-azidocarboxylic acid derivatives of the formula (III) with inversion of configuration (S_{N}2 reaction) has been described in the prior art for only a few very specific reactions. A great problem in this reaction is the undesired competing elimination of sulfonate to generate the alkene.

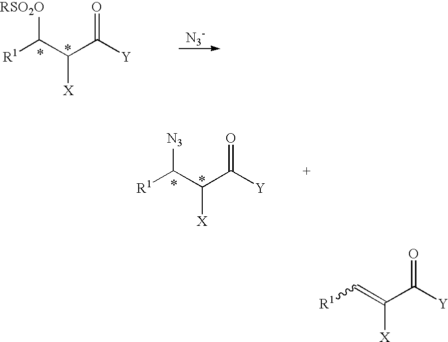

It is suspected that this side reaction occurs because the basic azide ion N₃⁻ can abstract the acidic hydrogen atom on the carbon atom in the α-position to the carbonyl group and the enolate formed stabilizes itself by eliminating the sulfonate RSO₃⁻. A corresponding loss of yield is the direct consequence.

An additional problem is the sometimes low stereoselectivity of the reaction in the conversion to the 3-azidocarboxylic acid derivatives of the general formula (III). This might be attributable, for example, to a Michael addition of the azide ion onto the alkene formed or else S_{N}1-like fractions in the substitution (both mechanisms lead to racemic product) or else to other effects.

The problem of low yields and/or inadequate stereoselectivities is distinctly less marked for those sulfonates of the general formula (II) in which R¹ and X form a ring than in the case of open-chain sulfonates of the general formula (II). For example, it should be mentioned that sulfonates of the general formula (II) in which R¹ and X form a four-membered ring have virtually no tendency for elimination to give the corresponding alkene, since the resulting cyclobutene derivative is virtually not formed due to the resulting high ring strain. Larger rings, however, allow a certain degree of undesired elimination.

The problem of low yields and/or inadequate stereoselectivities is particularly marked for sulfonates of the general formula (II) in which X is hydrogen (H), since this compound class has a strong tendency to eliminate to give the corresponding alkene.

In order to minimize the high tendency to eliminate with formation of the alkene and the problem of inadequate stereoselectivity in the direct azidation of sulfonates of optically active 3-hydroxycarboxylic acid derivatives of the general formula (II), reagents which strongly activate the OH group have been used in isolated cases in the prior art. Known particularly activating sulfonates are sulfonates with electron-withdrawing groups, for example trifluoromethane-sulfonates, p-nitrobenzenesulfonates or chlorobenzene-sulfonates.

J. Mulzer et al. describe the reaction of the para-nitrobenzenesulfonate of a 3-hydroxycyclopentanecarboxylic acid derivative with sodium azide in dimethylformamide (DMF) at room temperature in the form of a smooth S_{N}2 substitution to obtain the corresponding 3-azido ester in 95% yield [J. Mulzer et al., Synthesis 14, 2002, p. 2091-2095 and O. Langer et al., J. Org. Chem. 67, 2002, p. 6878-6883]. The combination of very good activation of the hydroxyl functionality as the para-nitrobenzenesulfonate (nosylate) and the presence of a substrate which has relatively low tendency to eliminate (X≠H, only one acidic hydrogen atom in the α-position; presence of a cycle: the elimination to give the corresponding cyclopentenecarboxylic ester is less critical in comparison to open-chain substrates) leads to a smooth reaction without noticeable formation of elimination products.

In contrast, according to Hoffman et al. the reaction of an open-chain nosylate of a methyl 3-hydroxycarboxylate (X═H) succeeds only with tetramethylguanidinium azide in methylene chloride [R. V. Hoffman et al., Tetrahedron 48(15), 1992, p. 3007-3020, synthesis of compound 3d]. However, the very expensive tetramethylguanidinium azide reagent is very disadvantageous for an industrial scale and economically viable performance of the process.

The reaction of a para-chlorobenzenesulfonate of a structurally very specific cyclic 3-hydroxycarboxylic acid derivative with sodium azide in DMSO succeeds at 65° C. in good yields of 78% and 81% [K. Ongania et al., Arch. Pharm. (Weinheim) 318, 1985, p. 2-10]. This success is attributable to the combination of the high activation of the hydroxyl functionality by means of a para-chlorobenzenesulfonate and especially to the substrate itself, which has virtually no tendency to eliminate (X≠H; presence of a four-membered ring (beta-lactam); the elimination to the corresponding alkene is virtually impossible). Moreover, for this reaction ten equivalents of the sodium azide reagent are required. This makes the described process uneconomic and expensive.

T. G. Hansson et al. describes the reaction of an open-chain trifluoromethanesulfonate of a 3-hydroxycarboxylic acid derivative (X═OR) with tetrabutylammonium azide in methylene chloride at low temperatures of −70° C. [T. G. Hansson et al., J. Org. Chem. 51, 1986, p. 4490-4492]. However, the very expensive tetrabutylammonium azide reagent and the very low temperatures are very disadvantageous for an industrial and economically viable performance of the process. Moreover, this publication points out that solutions of tetrabutylammonium azide in methylene chloride can form explosive products. A related reaction with comparable disadvantages is also described by R. Wagner et al. [R. Wagner et al., Synthesis 9, 1990, p. 785-786], which explicitly references the undesired eliminatio products obtained almost exclusively when less activating sulfonates are used.

The reaction of an open-chain trifluoromethanesulfonate of a 3-hydroxycarboxylic ester (X═F) with sodium azide in DMF at −5° C. leads to the corresponding azide in only 56% yield [F. B. Charvillon et al., Tetrahedron Lett. 37 (29), 1996, p. 5103-5106].

Sterically very strongly hindered trifluoromethanesulfonates of cyclic 3-hydroxycarboxylic esters based on an oxetane structure react with sodium azide in DMF to give the corresponding azides in yields of more than 90% [Y. Wang et al., Tetrahedron Lett. 32(13), 1991, p. 1675-1678 and S. F. Barker et al., Tetrahedron Lett. 42, 2001, p. 4247-4250]. Here too, only the combination of extremely good activation of the hydroxyl functionality by a trifluoromethanesulfonate and especially the presence of a substrate which in turn has virtually no tendency to eliminate (X≠H; presence of a four-membered ring (oxetane); the elimination to the corresponding alkene is virtually impossible) leads to a smooth reaction.

The processes described to date from the prior art all constitute reactions of highly activated sulfonates (e.g. triflates, chlorobenzenesulfonates and p-nitrobenzene-sulfonates) of very specific, usually cyclic 3-hydroxy-carboxylic acids. These processes are thus optimized for very specific substrates and do not offer any indications to a process usable broadly and on the industrial scale.

To date, the prior art has also not described any azidation process using alkali metal azides with strongly activating sulfonates of those substrates which have a distinct tendency to elimination, especially open-chain substrates of the general formula (II) in which X is hydrogen (H).

The great disadvantage of using highly activated sulfonates is the associated high costs. For example, activation as the trifluoromethanesulfonate is very expensive. However, this variant may also be viable in certain cases.

Of significantly greater industrial interest than the above-described, particularly useful activated sulfonates are aryl-, aralkyl-, alkenyl- and alkylsulfonates (for example toluenesulfonates, benzenesulfonates or methanesulfonates). This is connected to the fact that reagents for introducing these groups are available commercially on a large scale and very inexpensively. However, these so-called “non-activated” sulfonates are generally significantly less reactive and require more severe reaction conditions which can lead to undesired side reactions.

In principle, the activation of the OH group of optically active 3-hydroxycarboxylic acid derivatives as 3-halocarboxylic acid derivatives should be mentioned, which will be discussed briefly below. EP 1344763 A1 describes the reaction of optically active 3-halocarboxylic esters, especially 3-chlorocarboxylic esters, with alkali metal azides in water or a mixture of water and a water-soluble organic solvent. However, the use of temperatures of 94-96° C. over several hoursconstitute a safety problem in the preparation of the thermally sensitive and at least potentially explosive 3-azidocarboxylic esters. Moreover, although a very large excess of 10 equivalents of sodium azide is used, chemical yields of only 65.3% are achieved, for example, for methyl (3R)-azidobutanoate. The preparation of the optically active 3-halocarboxylic esters used is also complicated and expensive.

EP 1344763 A1 likewise discloses problems that exist in the reaction of tosylates of optically active methyl 3-hydroxybutyrate with sodium azide in various solvents or solvent mixtures. In principle, it is difficult in DMF to perform the reaction under mild conditions. However, very high temperatures are very disadvantageous for an industrial scale process, one reason being the stability of the product (organic azide). Moreover, it is very difficult to remover DMF from the product owing to the high boiling point.

In a biphasic solvent mixture of water/toluene, the achievable yield is very low and the products have an inadequate optical purity. When additional ethylene glycol is used, the conversions increase, but no significant improvement is achieved with regard to the stereoselectivity of the reaction.

The prior art discloses only a few additional reactions of “non-activated” sulfonates of optically active 3-hydroxycarboxylic acid derivatives of the general formula (II). Some of these reactions are again very substrate-specific

D. Seebach et al. describes the reaction of the tosylate of methyl (R)-3-hydroxybutyrate with sodium azide and subsequent hydrogenation to give the (S)-3-aminobutyric ester. However, no data on the specific experimental conditions are provided, especially with regard to solvent used, the stoichiometries, the reaction temperature employed, etc. Yield and optical purity of the 3-azido ester are likewise not published [D. Seebach et al., Tetrahedron Lett. 28(27), 1987, p. 3103-3106].

Park et al. describe, for example, the reaction of a tosylate of ethyl (R)-3-hydroxybutyrate with sodium azide in water under phase transfer catalysis with hexadecyltributylphosphonium bromide to give the corresponding (S)-azide in 76% or 78% yield [S. H. Park et al., J. Chem. Res. (S), 2001, p. 498-499; S. H. Park, Bull. Korean Chem. Soc. 24(2), 2003, p. 253-255]. However, the use of 10 mol % (based on the tosylate used) of the expensive hexadecyltributylphosphonium bromide reagent as a phase transfer catalyst makes the process uninteresting for industrial scale use.

Corey et al. reports the reaction of the mesylates of (α-methylated 3-hydroxymethyl esters (syn arrangement of the methyl and OMes groups) with tetrabutylammonium azide in acetone [E. J. Corey et al., Tetrahedron Lett. 32(39), 1991, p. 5287-5290]. Similar reactions of tosylates of α-methylated 3-hydroxycarboxamides (syn arrangement of the methyl and OTos groups) were performed using tetramethylguanidinium azide in CH₂Cl₂ or sodium azide/crown ether in DMF, although the yields of the azides at 54° C. and 30° C. are not suitable for an industrial scale performance of the process [J. Kimura et al., J. Org. Chem. 67, 2002, p. 1760-1767]. In general, the use of expensive reagents such as tetrabutylammonium azide, tetramethylguanidinium azide and crown ethers is industrially and economically very disadvantageous.

Ko et al. report the reaction of the tosylate of an α-hydroxy-substituted 3-hydroxyethyl ester (anti arrangement of the α-hydroxy and β-OTos groups) with sodium azide in boiling DMF (boiling point 153° C.) in 90% chemical yield [S. Y. Ko, J. Org. Chem. 67, 2002, p. 2689-2691]. The very high temperatures employed are, however, unsuitable for the industrial implementation of such a process, since organic azides are known to be thermally sensitive and a potential explosion risk.

Weigl et al. report the preparation of a structurally very unusual bicyclic α-amino-substituted 3-azido amide from the corresponding mesylate with sodium azide in DMF at 79% yield, the reaction likewise succeeding only at very high temperatures of 155° C. [M. Weigl et al., Bioorg. Med. Chem. 10, 2002, p. 2245-2257].

Kiss et al. (Synthesis 8, 2005, p. 1265-1268) report the reaction of the tosylates of cis-β-hydroxycycloheptane- and cis-β-hydroxycyclooctanecarboxylic esters with sodium azide in DMF at room temperature. However, the corresponding trans azides are obtained in only 36% and 34% yield respectively. In virtually identical portions, the corresponding alkene is obtained as the elimination product, which makes this process likewise uninteresting for industrial scale implementation.

Only 3-hydroxycarboxylic acid derivatives in which the competing elimination to give the alkene is ruled out by double substitution in the α-position to the carboxylic acid function (no acidic hydrogen atom present in the α-position to the carboxylic acid function), can be reacted via the mesylate with sodium azide in DMSO under comparatively mild conditions (T=80° C.) and in moderate to good yields (62-76%) to give the corresponding azide [M. J. Burke et al., Tetrahedron: Asymmetry 11, 2000, p. 2733-2739].

In summary, the greatest disadvantages of the prior art processes for direct azidation of sulfonates of 3-hydroxycarboxylic acid derivatives of the general formula (II) to obtain compounds of the general formula (III) include:

a) the use of very expensive reagents, such as the use of phosphonium salts as phase transfer catalysts, the use of alkylammonium azides and the use of crown ethers,

b) the desired products being obtained only with low chemical and/or optical yields,

c) optimization of the process only very specifically to certain substrates (low substrate range) and

d) the use of very high temperatures which are unsuitable for the labile products and can lead to safety problems, or else the use of very low temperatures which can be realized on the industrial scale only at great cost and inconvenience.

### SUMMARY OF THE INVENTION

It is thus an object of the invention to provide an alternative process for azidating compounds of the general formula (II) which overcomes the disadvantages from the prior art. It is a particular object to provide a process with which optically active 3-azidocarboxylic acid derivatives of the general formula (III) and the optically active 3-aminocarboxylic acid derivatives of the general formula (IV) obtainable therefrom by reduction can be prepared particularly inexpensively and in an economically viable manner from the corresponding optically active 3-hydroxycarboxylic acid derivatives of the general formula (I) in high yields, high stereoselectivities and in a process performable advantageously on the industrial scale.

In an embodiment of the invention, the object is achieved by performing the azidation in the presence of specific solvents or solvent mixtures. The process of this embodiment comprises reacting sulfonates of the general formula (II)

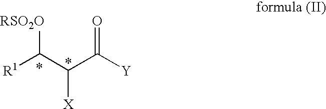

wherein:R is a linear or branched, saturated or unsaturated alkyl, aryl or aralkyl radical which is cyclic or contains cyclic groups and is optionally substituted by Q
with an alkali metal azide, which comprises effecting the reaction in a solvent selected from the group comprising carboxamides of the general formula (V)

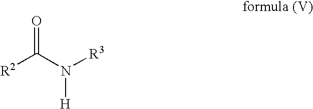

wherein
R² and R³ are each independently hydrogen or a linear or branched, saturated or unsaturated alkyl, aryl or aralkyl radical which is cyclic or contains cyclic groups and is optionally substituted by Q; a solvent mixture which comprises carboxamides of the general formula (V); a solvent mixture composed of water and a solvent miscible homogeneously with water; water with the proviso that the addition of a phase transfer catalyst is avoided in the case of reaction in water; and DMSO.

### DETAILED DESCRIPTION OF THE PREFERRED EMBODIMENT(S)

In an embodiment of the present invention, a process for enantioselectively preparing 3-azidocarboxylic acid derivatives of the general formula (III) is provided:

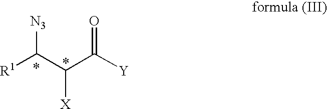

wherein: R¹ is a linear or branched, saturated or unsaturated alkyl, aryl or aralkyl radical which is cyclic or contains cyclic groups and is optionally substituted by Q, or is a carboxylate or carboxamide group; Q is selected from the group comprising carboxylato, carboxamido, halogen, cyano, nitro, acyl, silyl, silyloxy, aryl, heteroaryl, OR′, NR′R″ and SR′, where R′ and R″ are each independently hydrogen, a linear or branched, saturated or unsaturated alkyl, aryl or aralkyl radical which is cyclic or contains cyclic groups and is optionally substituted by Q, or a suitable protecting group;
X is hydrogen or a radical as defined for R¹ or a substituent as defined for Q;
Y is a radical selected from the group comprising OR′, NR′R″ and SR′, where R′ and R″ are each independently hydrogen, a suitable protecting group or a linear or branched, saturated or unsaturated alkyl, aryl or aralkyl radical which is cyclic or contains cyclic groups and is optionally substituted by Q,
where R¹ and X are optionally joined to one another and may form an at least 5-membered ring.

The process of this embodiment comprises reacting sulfonates of the general formula (II)

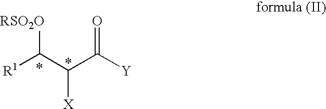

wherein:R is a linear or branched, saturated or unsaturated alkyl, aryl or aralkyl radical which is cyclic or contains cyclic groups and is optionally substituted by Q
with an alkali metal azide, which comprises effecting the reaction in a solvent selected from the group comprising carboxamides of the general formula (V)

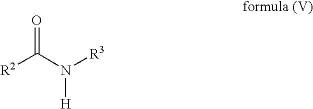

wherein
R² and R³ are each independently hydrogen or a linear or branched, saturated or unsaturated alkyl, aryl or aralkyl radical which is cyclic or contains cyclic groups and is optionally substituted by Q; a solvent mixture which comprises carboxamides of the general formula (V); a solvent mixture composed of water and a solvent miscible homogeneously with water; water with the proviso that the addition of a phase transfer catalyst is avoided in the case of reaction in water; and DMSO.

The invention further provides for the further reaction of the resulting 3-azidocarboxylic acid derivatives of the general formula (III) in a downstream step by means of reduction to give 3-aminocarboxylic acid derivatives of the general formula (IV)

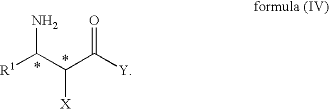

Compounds of the general formula (I), (II), (III) and (IV) are characterized by the presence of two chiral centers (when X is not hydrogen) or one chiral center (when X is hydrogen) in the structural section shown in formula (IV) which are each marked with * in the general formulae (I), (II), (III) and (IV) and may each be present either in the R or in the S form. The reaction in the process according to the invention proceeds with virtually full stereoselectivity (complete inversion at the carbon atom adjacent to R¹), so that, when optically active reactants are used, especially the compounds of the general formula (II), optically active products, especially of the general formula (III), are obtained.

Therefore, when enantiomerically pure or enantiomerically enriched reactants are used, the process according to the invention in turn affords enantiomerically pure or enantiomerically enriched products of the opposite configuration in each case at the carbon atom adjacent to R¹. It is thus possible to use compounds of the general formula (II) in R,R, S,R, R,S and S,S configuration (cf. formulae (IIa-d)), the chiral carbon atom adjacent to R¹ being subjected to an inversion.

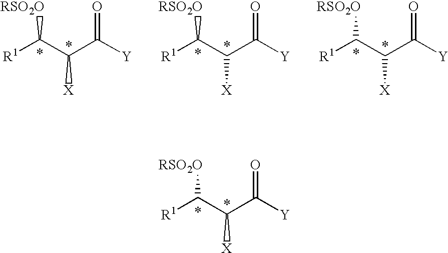

formula (IIa) formula (IIb) formula (IIc) formula (IId)
It has been found that, surprisingly, optically active 3-azido-carboxylic acid derivatives of the general formula (III) can be obtained with particularly high chemical purities and high optical yields by reacting sulfonates of the general formula (II) of optically active 3-hydroxycarboxylic acids of the general formula (I) with alkali metal azides when the inventive solvents or solvent mixtures are used.

The corresponding optically active 3-aminocarboxylic acid derivatives of the general formula (IV), which can be prepared by reduction from the corresponding optically active 3-azidocarboxylic acid derivatives of the general formula (III), are thus obtainable in this process, likewise in high yield and high optical purity, by this additional step.

R¹ and X may be joined together and form a cycle consisting of at least 5 atoms. Preferably, R¹ and X may be joined together and form a cycle consisting of at least 5 atoms 5-10 atoms. More preferably, R¹ and X may be joined together and form a cycle consisting 6-10 atoms, Such rings may include carbon atoms, Optionally, such rings may also contain heteroatoms, especially heteroatoms selected from the group comprising oxygen, nitrogen, sulfur and phosphorus. The reactants used in the process according to the invention are more preferably open-chain compounds of the general formula (II) in which R¹ and X are not joined together.

Compounds of the general formula (II) in which R¹ is an aryl radical optionally substituted by Q (e.g. phenyl) generally have a very strong tendency to react to give the corresponding alkene with elimination of sulfonate.

In the process according to the invention, preference is given to reacting compounds of the general formula (II) in which R¹ is a linear or branched, saturated or unsaturated alkyl or aralkyl radical which is cyclic or contains cyclic groups and is optionally substituted by Q. More preferably, R¹ contains from 1 to 10 carbon atoms. Preference is likewise given to carboxylate and carboxamide radicals for R¹. Possible specific embodiments thereof are methyl carboxylate, ethyl carboxylate, propyl carboxylate, isopropyl carboxylate, n-butyl carboxylate, isobutyl carboxylate, tert-butyl carboxylate and benzyl carboxylate, or N-methylcarboxamide, N-ethylcarboxamide, N-propylcarboxamide, N-isopropylcarboxamide, N-n-butyl-carboxamide, N-isobutylcarboxamide, N-tert-butylcarboxamide and N-benzylcarboxamide.

Q is preferably selected from the group comprising carboxylato, carboxamido, halogen, OR′, NR′R″ and SR′, where R′ and R″ are each independently hydrogen or a radical as defined for R¹ or a suitable protecting group.

The substituent R¹ is more preferably selected from the group comprising linear or branched, saturated or unsaturated C1-C10 alkyl or aralkyl radicals which are cyclic or contain cyclic groups, especially methyl, ethyl, propyl, isopropyl, n-butyl, isobutyl, tert-butyl, pentyl, hexyl, heptyl, octyl, nonyl and benzyl.

When R′ and R″ represent a protecting group, this may in particular be a hydroxyl protecting group or thio protecting group, or an amine protecting group.

Among the hydroxyl and thio protecting groups, it is possible to select from all protecting groups suitable to the person skilled in the art and known for this purpose; a selection of suitable OH protecting groups is described in particular in T. W. Greene, P. G. M. Wuts, “Protective Groups in Organic Synthesis”, 2nd Edition, Wiley 1991, p. 10-117.

The hydroxyl protecting groups are preferably selected from the group comprising acyl radicals, alkyl radicals, alkoxyalkyl radicals, arylalkyl radicals, arylalkoxyalkyl radicals or silyl radicals. Particular preference is given to protecting groups from the group comprising benzoyl, n-butyryl, isobutyryl (2-methylpropionyl), pivaloyl, propionyl and acetyl, methyl, ethyl and propyl, methoxymethyl, 1-ethoxyethyl and 2-methoxyethoxymethyl, benzyl, 4-methoxybenzyl and triphenylmethyl, benzyloxymethyl and 4-methoxybenzyloxymethyl, trimethylsilyl, triethylsilyl, triisopropylsilyl, tert-butyldimethylsilyl and tert-butyldiphenylsilyl.

A selection of suitable amino protecting groups can be taken by the person skilled in the art from T. W. Greene, P. G. M. Wuts, “Protective Groups in Organic Synthesis”, 2nd Edition, Wiley 1991, p. 309-385. The amino protecting groups used are preferably acyl radicals, acyloxycarbonyl radicals, alkyl radicals, arylalkyl radicals or silyl radicals. Preference is given to selecting protecting groups from the group comprising benzoyl, acetyl and formyl, tert-butyloxycarbonyl (BOC), 9-fluorenylmethyloxycarbonyl (Fmoc) and benzyloxycarbonyl (Z), methyl and allyl, benzyl and 4-methoxybenzyl, trimethylsilyl, triethylsilyl, triisopropylsilyl, tert-butyldimethylsilyl and tert-butyldiphenylsilyl.

In the process according to the invention, preference is given to reacting compounds of the general formula (II) in which Y is OR′ and is thus a carboxylic ester derivative, since the corresponding optically active 3-hydroxycarboxylic esters which serve as the starting compound are obtainable industrially in a particularly simple and inexpensive manner.

Preferred radicals for R′ and R″ are selected from the group comprising hydrogen, methyl, ethyl, n-propyl, isopropyl, n-butyl, isobutyl, tert-butyl, pentyl, hexyl, heptyl, octyl, nonyl, benzyl, phenyl, naphthyl, acyl and silyl.

X may generally be hydrogen or a substituent as defined for R¹ or a substituent as defined for Q, especially from their preferred embodiments. X is preferably methyl, ethyl, n-propyl, isopropyl, n-butyl, isobutyl, tert-butyl, pentyl, hexyl, heptyl, octyl, nonyl, benzyl, halogen, OR′, NR′R″ and SR′, where R′ and R″ are each independently hydrogen or a linear or branched, saturated or unsaturated, alkyl, aryl or aralkyl radical which is cyclic or contains cyclic groups and is optionally substituted by Q, or represent a suitable protecting group, especially hydrogen, methyl, ethyl, n-propyl, isopropyl, n-butyl, isobutyl, tert-butyl, pentyl, hexyl, heptyl, octyl, nonyl, benzyl.

In a particularly preferred embodiment of the process according to the invention, compounds of the general formula (II) in which X is hydrogen (H) are used. The substance class of optically active 3-azidocarboxylic acid derivatives of the general formula (III) obtainable therefrom, where X is H and which contain a chiral carbon atom adjacent to R¹, are particularly valuable, since these compounds are obtainable only with difficulty by means of the processes known from the prior art, since the corresponding starting compounds of the general formula (II) where X═H have, inter alia, a great tendency to undesired elimination to form the corresponding alkene. Moreover, the industrially and economically particularly interesting optically active 3-aminocarboxylic acid derivatives of the general formula (IV) in which X is H are obtainable from 3-azidocarboxylic acid derivatives of the general formula (III) in which X is H by means of subsequent reduction.

A great advantage of the process according to the invention is also the surprising fact that, the best results can be achieved in the reaction with alkali metal azides, especially sodium azide. Those carboxylic acid derivatives of the general formula (II) in which X is H, especially including their esters (Y is OR′), which have a very particular tendency toward undesired elimination are a specific example. This observation completely contradicts the teaching available to the person skilled in the art from the prior art.

The corresponding optically active 3-hydroxycarboxylic acid derivatives of the general formula (I) in which X is H, especially including their esters (Y is OR′) are also obtainable industrially in a particularly simple and inexpensive manner.

In the process according to the invention, preference is given to reacting compounds of the general formula (II) in which R is a linear or branched alkyl, aryl or aralkyl radical optionally substituted by Q.

Owing to the relatively high costs of preparation (e.g., trifluoromethanesulfonates, p-nitrobenzenesulfonates, and chlorine-substituted benzenesulfonates), particular preference is given to using toluene-, benzene-, alkyl- and aralkylsulfonates. For example, these sulfonates can be obtained from the optically active 3-hydroxycarboxylic acid derivatives of the general formula (I) and the industrially very inexpensively available toluenesulfonyl chlorides, benzenesulfonyl chlorides, alkyl- and aralkylsulfonyl chlorides, or toluenesulfonic anhydrides, benzenesulfonic anhydrides, alkyl- and aralkylsulfonic anhydrides.

More preferabley, compounds of the general formula (II) in which R is a linear or branched alkyl radical as used. A specific particularly preferred embodiment is methanesulfonic esters which can, for example, be prepared very inexpensively from the optically active 3-hydroxycarboxylic acid derivatives and methanesulfonyl chloride in the presence of a base (see also example 1) in high yield in a process which is very simple to perform industrially.

The alkylsulfonic esters, especially those in which R is a lower alkyl radical, such as methyl, ethyl, n-propyl, isopropyl or butyl, also have the advantage that the protecting group contributes to the molar mass of the sulfonate only to a relatively low degree and hence does not unnecessarily increase the mass of this intermediate. Furthermore, in the reaction of the corresponding sulfonates of the general formula (II) with alkali metal azides, the by-product eliminated is an alkylsulfonate. For example methanesulfonate, which has a relatively low molar mass, is of particular interest especially with regard to the avoidance of unnecessary wastes. Furthermore, compounds of the general formula (II) in which R is an alkyl radical, especially a methyl radical, are particularly suitable with regard to their stability, handling and reactivity.

The azide sources used are generally metal azides MN₃ in which M is an alkali metal. Particular preference is given to using sodium azide which is available industrially in large amounts and relatively inexpensively.

In general, 1-20 equivalents of alkali metal azide based on the sulfonate of the general formula (II) are used. Preferably, 1-5 equivalents equivalents of alkali metal azide based on the sulfonate of the general formula (II) are used. More preferably, 1-2 equivalents equivalents of alkali metal azide based on the sulfonate of the general formula (II) are used.

A further advantage of the process according to the invention is thus that the use of expensive azide reagents and the use of high excesses is avoided.

The process according to the invention is preferably carried out within a temperature range of from −40° C. up to the boiling point of the solvent or solvent mixture. The reaction is preferably effected at a temperature of from 0° C. to 100° C., most preferably within a range of from 20° C. to 80° C.

Since the products of the general formula (III), being organic azides, are potentially thermally sensitive and/or an explosion risk, the reaction is generally performed at minimum temperature, but at which an acceptable reaction rate and a good yield and quality of the product still result. Thus, it may be advantageous depending on the substrate also to use relatively expensive sulfonates of the general formula (II), especially trifluoromethanesulfonates or substituted benzenesulfonates, which have a significantly increased reactivity in comparison to methanesulfonates. It may also be particularly advantageous in this context to employ high temperatures and to balance out the potential risks associated with them by virtue of high reaction rates enabling, for example, the performance of a continuous process with short residence time and a resulting minimization of the reactor volume with simultaneously high throughput.

An increase in the reaction temperature can also lead to an improved result with regard to the achievement of higher yields and/or better selectivities, since this can significantly favor the S_{N}2 substitution of sulfonate by the azide ion in comparison to side reactions, especially elimination. The optimal temperature for one substrate also depends significantly upon the reactivity of the sulfonate used, especially owing to the significantly different reactivities of, for example, trifluoromethanesulfonates and methanesulfonates.

A characterizing feature of the process according to the invention is that the reaction is effected in the presence of
a) carboxamides of the general formula (V)

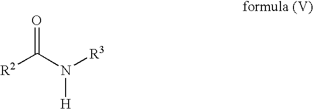

wherein
R² and R³ are each independently hydrogen or a linear or branched, saturated or unsaturated alkyl, aryl or aralkyl radical which is cyclic or contains cyclic groups and is optionally substituted by Q;
b) a solvent mixture which comprises carboxamides of the general formula (V) mentioned under a);
c) a solvent mixture of water and a solvent miscible homogeneously with water;
d) DMSO; or
e) water, combined with the proviso that, in the case of the reaction in water, the addition of a phase transfer catalyst, especially the addition of hexadecyltributylphosphonium bromide, as reported by Park et al., is avoided.

In example 2, the reaction of the optically active methanesulfonic ester of (R)-ethyl 3-hydroxybutyrate ((R)-EHB mesylate) with sodium azide to give the corresponding optically active (S)-ethyl 3-azidobutyrate ((S)-EHB azide) in various solvents or solvent mixtures is compared (cf. table 1).

In DMF, which is the solvent most frequently used for such a reaction in the prior art, a yield of only 37% of the product is obtained with a very poor enantiomeric excess (ee) of only 74% ee. The main product formed in DMF is the corresponding alkene by elimination at 57% (table 1, line 1), which makes this process unviable for an industrial scale reaction.

Even in DMSO as the solvent (table 1, line 2), a significantly improved result is achieved under mild conditions (70% yield, 21% alkene, 98% ee). Even comparative example 5 shows the distinct superiority of DMSO in comparison to DMF as the solvent.

In dimethoxyethane as the solvent (table 1, line 3), virtually no conversion to the product with low elimination to give the alkene is observed, and such a process is thus fundamentally unviable.

In a biphasic mixture of water and toluene (table 1, line 4), only 28% product, 20% alkene and, by hydrolysis of the methanesulfonic ester, also 13% of the free alcohol (ethyl 3-hydroxybutyrate) are obtained. This process too is unsuitable for industrial scale implementation.

Surprising, it has been found that very good results are obtained solely in water or a solvent mixture composed of a solvent miscible homogeneously with water and water (table 1, lines 5-9). Thus, even in DMF, as a result of the addition of water, a sharp rise in the achievable yields and the enantio-selectivity can be detected (table 1, line 6). The yield in DMSO can also be enhanced even further by the addition of water compared to the use of pure DMSO (table 1, line 7). The product is generated with very good enantiomeric excesses of in some cases above 98% and in yields of generally more than 75% under mild conditions. The proportion of the elimination product is reduced throughout to less than 10%.

When water alone is used as the solvent, the process according to the invention avoids the addition of phase transfer catalyst, especially of an alkylphosphonium salt such as hexadecyltributylphosphonium bromide. Surprisingly, in spite of avoiding the addition of these compounds, comparable yields with high enantiomeric excesses are achieved, as reported by Park et al.

In the presence of larger amounts of water (e.g. 50% content in the solvent mixture), slight hydrolysis of the sulfonate to the corresponding alcohol is observed under some circumstances depending on the reaction conditions and the substrate used (see table 1, lines 5-9), so that, in such cases, a different inventive solvent is preferable or the content of water in the solvent mixture is reduced.

In general, all mixing ratios of water and the solvents miscible homogeneously with water are possible. Typically, mixing ratios of water to solvent from 100 to 0.01:1 are used. Preferably, mixing ratios of water to solvent from 10 to 0.1:1 are used, More preferably, mixing ratios of water to solvent from 2 to 0.5:1 are used. Most preferably, a mixing ratio of water to solvent of 1:1 is used.

The solvents miscible homogeneously with water used may generally be organic solvents which have no solubility/miscibility gaps with water. These solvents are preferably selected from the group comprising alcohols, acetone, dimethyl sulfoxide (DMSO), dimethylformamide (DMF), or amides of the general formula (V), especially formamide or N-methylformamide (NMF). Particular preference is given to using dimethyl sulfoxide (DMSO), dimethylformamide (DMF), or amides of the general formula (V), especially formamide or N-methylformamide (NMF).

A particularly surprising finding is that, when carboxamides of the general formula (V) are used as solvents, particularly good results can be achieved (table 1, lines 10-15; examples 3 and 4). This is particularly surprising because dimethylfbrmamide, which is structurally closely related to the compounds of the general formula (V), provides a very poor result as the solvent in the same reaction (table 1, line 1).

Thus, even in N-methylformamide (NMF) as the solvent, a significantly improved result is achieved (table 1, line 12). In comparison to DMF, the content of the alkene elimination product is reduced drastically from 57% to 16%. The enantiomeric excess likewise rises drastically from 74% ee to above 99% ee.

Even better results can be achieved with formamide as the solvent (table 1, lines 10-11 and 13-15). (S)-Ethyl 3-azidobutyrate can be prepared in yields of over 90% with an enantiomeric excess of over 99%. The fractions of alkene and free alcohol formed are negligibly low.

Preferred solvents from the class of the amides of the general formula (V) are those in which R² and R³ are each independently selected from the group comprising hydrogen, alkyl, aryl and aralkyl radicals. Among the hydrocarbon radicals, linear, branched or cyclic C1 to C10 hydrocarbon radicals are typically used. R² and R³ are more preferably each independently selected from the group comprising hydrogen, methyl, ethyl, n-propyl, i-propyl, n-butyl, isobutyl, tert-butyl, pentyl, hexyl, heptyl, octyl, nonyl, benzyl. The amides of the general formula (V) used are more preferably formamide, acetamide or N-methylformamide (NMF). Most preferably, amides in which R³ is hydrogen, such as acetamide and formamide are used, especially formamide.

These solvents, especially formamide or N-methylformamide, may be used alone or in a mixture with water or other solvents miscible, partly miscible or even immiscible with them. The content of the amide of the general formula (V) in such a mixture should generally be more than 10%. Preferably, the content of the amide of the general formula (V) in such a mixture is more than 50%. More preferably, the content of the amide of the general formula (V) in such a mixture is more than 90%. Most preferably, the content of the amide of the general formula (V) in such a mixture is more than 95%. However, in principle, all conceivable mixing ratios may be possible and viable depending on the specific circumstances. Apart from water, useful solvents are, for example, also compounds selected from the class of the alcohols, ethers, ketones, halogenated hydrocarbons, carboxylic esters, aromatic hydrocarbons and alkanes. Specific examples include acetone, DMSO, toluene, methyl tert-butyl ether, diethyl ether, methylene chloride and ethyl acetate. Depending on the specific circumstances, advantages in the reaction, the yields, the qualities or with regard to the safe performance of the reaction can in some cases be achieved with such mixtures.

In spite of only a slight change in the molecular structure of the solvent used, comparison of the solvents DMF, NMF and formamide shows a drastic dependence of the product distribution. This is a very surprising result that could not have been expected by the person skilled in the art.

It is evident from examples 3, 4 and 5 that other optically active 3-azidocarboxylic acid derivatives of the general formula (III) are also obtainable by the process according to the invention in high yields and high enantiomeric excesses under mild conditions.

The isolation of the products from the reaction mixture can be effected by the customary methods of preparative organic chemistry. For example, an aqueous organic workup of the reaction mixture is advantageous. Excess sodium azide and other water-soluble components (for example water-soluble solvents, 3-hydroxycarboxylic acid derivatives present in the reaction mixture when they have sufficient solubility in the aqueous phase) remain for the most part in the aqueous phase. The product can be extracted from the organic phase with organic solvents (for example with ethyl acetate, ether, toluene, methylene chloride). The product is usually already present in high purity in the extracted solution. However, for example, it is also possible to subject the reaction mixture without further workup directly to hydrogenation or reduction of the azide to the amine.

In a preferred embodiment of the process according to the invention, the workup, in the case of carboxamides of the general formula (V) (e.g. formamide) as the solvent, is effected by means of a biphasic system. For example, formamide forms biphasic mixtures with aromatics such as toluene or benzene, ethers such as methyl tert-butyl ether (MTBE), chlorinated hydrocarbons such as methylene chloride or alkanes such as pentane or petroleum ether. By means of the solvents added in the workup, virtually full extraction of the product from the reaction medium is achieved. For example, in the preparation of (S)-ethyl 3-azidobutyrate, quantitative extraction is achieved with toluene from formamide as the solvents used for the reaction. In addition, it is also possible to add the extractant (e.g. toluene) actually at the start, and to perform the reaction in a biphasic mixture of, for example, formamide and toluene (cf. example 6); the by-products are almost fully removed from the product in the extraction and the toluene phases already comprises the organic azide in very pure form. In addition to small traces of impurities, the formamide phase comprises mainly excess sodium azide, so that optional metered addition of alkali metal azide, especially sodium azide, allows the formamide phase to be used again for azidation of a compound of the general formula (II), especially of a mesylate.

Generally, the products of the general formula (III) obtainable by the process according to the invention are preferably handled in solution and not isolated as a pure substance, since the risk potential of the potentially thermally sensitive and explosive azides, especially in the course of industrial scale handling, can thus be significantly reduced.

Usually, the azides of the general formula (III) are reduced with a reducing agent, directly and without isolation of the azide as a pure substance, to give the optically active amines of the general formula (IV), for which the standard processes can be used. The corresponding optically active 3-aminocarboxylic acid derivative can be prepared with hydrogen, for example, with catalysis by Pd/C (cf. example 7).

Accordingly, the optically active 3-aminocarboxylic acid derivatives of the general formula (IV), which can be obtained by reduction from the corresponding optically active 3-azidocarboxylic acid derivatives of the general formula (III), are also advantageously obtainable in high yield and high optical purity by the process according to the invention.

The examples which follow serve to illustrate the invention in detail and are in no way to be interpreted as a restriction.

### EXAMPLES

### Example 1

### Preparation of the methanesulfonic ester of ethyl (R)-3-hydroxybutyrate

20 g of ethyl (R)-3-hydroxybutyrate (151 mmol, ee>99%) are dissolved in 100 ml of dichloromethane at 0° C. and admixed with 24.1 ml of triethylamine (174 mmol). At 0° C., a solution of 12.9 ml of methanesulfonyl chloride (166 mmol) in 11 ml of dichloromethane is added dropwise, in such a way that the temperature does not exceed 150° C. The mixture is stirred at 20° C. for a further 1 h. The reaction solution is admixed with saturated sodium hydrogencarbonate solution and stirred, and the organic phase is removed and washed with water. After the solvent has been removed, the methanesulfonic ester is dried at 40° C. under reduced pressure.

Yield: 30.9 g (97%), content: 95%

The methanesulfonic esters of (R)-tert-butyl 3-hydroxybutyrate, (R)-methyl 3-hydroxypentanoate and (2R,3R)-ethyl 2-butyl-3-hydroxybutyrate can be prepared in a similar manner.

### Example 2

### General Experimental Method for Reacting the methanesulfonic ester of (R)-ethyl 3-hydroxybutyrate ((R)-EHB mesylate) with sodium azide

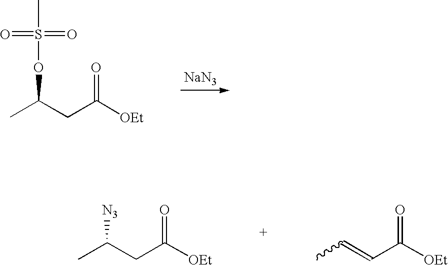

1 g of (R)-EHB mesylate (ee >99%, content: 95%, 3% alkene, 2% (R)-EHB) is admixed in K ml of the solvent or solvent mixture A with B equivalents of sodium azide (based on the mesylate used) and stirred at the temperature C specified for D hours. The reaction mixture is analyzed by means of gas chromatography for reactant content E, product content F, content of the alkene G, content of the ethyl 3-hydroxybutyrate (EHB) H and the enantiomeric excess I of the (S)-ethyl 3-azidobutyrate (see table 1).

TABLE 1

| | A | K [ml] | B [eq.] | C [° C.] | D [h] | E [%] | F [%] | G [%] | H [%] | I [%] |
|---|---|---|---|---|---|---|---|---|---|---|
| 1*⁾ | DMF | 5 | 2 | 40 | 11 | <1 | 37 | 57 | 4 | 74 |
| 2 | DMSO | 5 | 2 | 40 | 11 | <1 | 70 | 21 | 6 | 98 |
| 3*⁾ | DME | 5 | 2 | 40 | 13 | 85 | 2 | 9 | 2 | n.d. |
| 4*⁾ | H₂O/toluene | 2.5/2.5 | 2 | 40 | 13 | 7 | 28 | 20 | 13 | n.d. |
| 5 | H₂O/formamide | 2.5/2.5 | 2 | 40 | 13 | <1 | 78 | 2 | 17 | 94 |
| 6 | H₂O/DMF | 2.5/2.5 | 2 | 40 | 13 | <1 | 77 | 5 | 11 | 97 |
| 7 | H₂O/DMSO | 2.5/2.5 | 4 | 40 | 13 | <1 | 86 | <5 | 9 | >98 |
| 8 | H₂O | 5 | 2 | 40 | 6 | 11 | 69 | 10 | 10 | >98 |
| 9 | H₂O | 2.5 | 2 | 60 | 13 | <1 | 79 | 5 | 8 | >98 |
| 10 | Formamide | 2.5 | 2 | 40 | 13 | <1 | 86 | 5 | 2 | >99 |
| 11 | Formamide | 5 | 4 | 40 | 2 | <1 | 92 | 6 | 2 | >99 |
| 12 | NMF | 5 | 4 | 40 | 13 | <1 | 75 | 16 | 1 | >99 |
| 13 | Formamide | 5 | 2 | 60 | 2 | <1 | 90 | 6 | 3 | >99 |
| 14 | Formamide | 2.5 | 1.2 | 60 | 4 | <1 | 92 | 2 | 5 | >99 |
| 15 | Formamide | 2.5 | 1.2 | 100 | 0.5 | <1 | 88 | 4 | 6 | >99 |

Abbreviations:
DMSO = dimethyl sulfoxide;
DMF = dimethylformamide;
DME = dimethoxyethane;
NMF = N-methylformamide;
n.d. = not determined;
legend:
A solvent or solvent mixture; K amount of solvent;
B equivalents of sodium azide (based on mesylate);
C temperature;
D reaction time;
E reactant content;
F product content;
G content of the alkene;
H content of ethyl 3-hydroxybutyrate (EHB);
I enantiomeric excess of (S)-ethyl 3-azidobutyrate;
*)comparative examples

### Example 3

### Reaction of the methanesulfonic ester of (R)-tert-butyl 3-hydroxybutyrate ((R)-BHB mesylate) with sodium azide

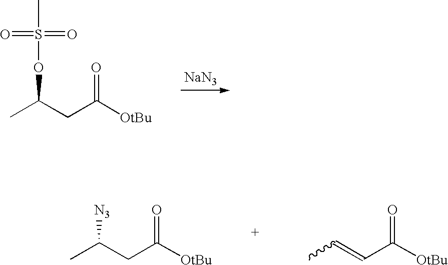

1 g of (R)-BHB mesylate (ee >99%, content: 95%) is dissolved in 2.5 ml of formamide, admixed with 2 equivalents of sodium azide (based on the mesylate used) and stirred at 60° C. for 6 h. The reaction mixture contains <1% reactant (mesylate), 95% product (azide), 2% alkene, 2% tert-butyl 3-hydroxybutyrate. The enantiomeric excess of the (S)-tert-butyl 3-azidobutyrate is >99%.

### Example 4

### General Experimental Method for the Reaction of the methanesulfonic ester of (R)-methyl 3-hydroxypentanoate ((R)-MHP mesylate) with sodium azide

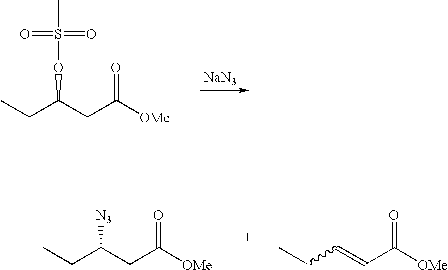

1 g of (R)-MHP mesylate (ee >99%, content: >95%) is admixed in K ml of the solvent or solvent mixture A with B equivalents of sodium azide (based on the mesylate used) and stirred at the temperature C specified for D hours. The reaction mixture is analyzed by means of gas chromatography for reactant content E, product content F, content of the alkene G, content of the methyl 3-hydroxypentanoate H and the enantiomeric excess I of the (S)-methyl 3-azidopentanoate (see table 2).

TABLE 2

| | A | K [ml] | B [eq.] | C [° C.] | D [h] | E [%] | F [%] | G [%] | H [%] | I [%] |
|---|---|---|---|---|---|---|---|---|---|---|
| 1 | Formamide | 5 | 4 | 40 | 2 | <1 | 93 | 5 | 1 | >98 |
| 2 | H₂O | 5 | 4 | 40 | 7 | <1 | 91 | 3 | 4 | >98 |
| 3 | H₂O/DMSO | 2.5/2.5 | 4 | 40 | 7 | <1 | 89 | 6 | 4 | >98 |
| 4 | Formamide | 5 | 2 | 60 | 4 | <1 | 89 | 7 | 2 | >99 |

Abbreviations:
DMSO = dimethyl sulfoxide;
legend:
A solvent or solvent mixture;
K amount of solvent or solvent mixture;
B equivalents of sodium azide (based on mesylate used);
C temperature;
D reaction time;
E reactant content;
F product content;
G content of the alkene;
H content of methyl 3-hydroxypentanoate;
I enantiomeric excess of (S)-methyl 3-azidopentanoate

### Example 5

### General Experimental Method for the Reaction of the methanesulfonic ester of (2R,3R)-ethyl 2-butyl-3-hydroxy-butyrate ((2R,3R)-butyl-EHB mesylate) with sodium azide

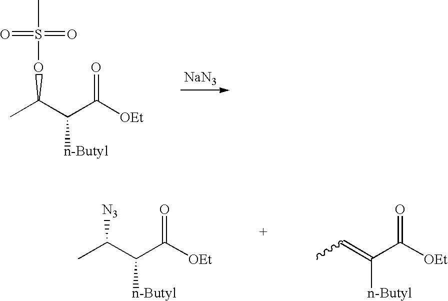

1 g of (2R,3R)-butyl-EHB mesylate (content: >95%) is admixed in K ml of the solvent A with B equivalents of sodium azide (based on the mesylate used) and stirred at the temperature C specified for D hours. The reaction mixture is analyzed by means of gas chromatography for reactant content E, product content ((2R,3S)-ethyl 2-butyl-3-azidobutyrate) F and content of the alkene G (see table 3).

TABLE 3

| | A | K [ml] | B [eq.] | C [° C.] | D [h] | E [%] | F [%] | G [%] |
|---|---|---|---|---|---|---|---|---|
| 1 | DMF | 5 | 2 | 60 | 14 | 6 | 70 | 18 |
| 2 | DMSO | 5 | 2 | 60 | 10 | 3 | 80 | 9 |

Abbreviations:
DMSO = dimethyl sulfoxide;
DMF = dimethylformamide

### Example 6

### Preparation and isolation of (S)-ethyl 3-azido-butyrate

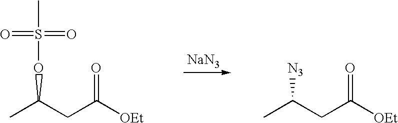

10 g of (R)-EHB mesylate (ee >99%, content: >95%) are dissolved in 25 ml of formamide, admixed with 1.2 equivalents of sodium azide (based on the mesylate used) and stirred at 60° C. for 4 h. The reaction mixture contains <1% reactant (mesylate), 92% product (EHB azide), 2% alkene and 5% ethyl 3-hydroxybutyrate. Extraction is effected 2× with 25 ml of toluene each time. This extracts the product quantitatively into the toluene phase. Sodium azide, alkene and ethyl 3-hydroxybutyrate remain virtually fully in the formamide phase. The yield is 6.8 g (92%). The purity of the product in the toluene phase is >97%. The enantiomeric excess of the (S)-ethyl 3-azidobutyrate is >99%.

It is, for example, also possible to add 25 ml of toluene actually at the start of the reaction and to perform the reaction of (R)-EHB mesylate with sodium azide in a biphasic mixture of formamide and toluene. Product yield and quality here are comparable with abovementioned results.

### Example 7

### Hydrogenation of (S)-ethyl 3-azidobutyrate to (S)-ethyl 3-aminobutyrate

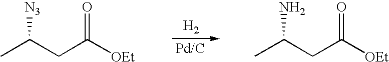

The toluene solution (approx. 50 ml) of the (S)-ethyl 3-azido-butyrate from example 6 is hydrogenated with hydrogen under catalysis with Pd/C (300 mg, 5%) at 20 bar at RT for 2 h. It is also possible to perform the hydrogenation at only 2 bar. Optionally, it is also possible to add a cosolvent, for example methanol. After filtration and washing of the filtercake with 10 ml of toluene, the product is obtained in quantitative yield (as a solution in 60 ml of toluene). The enantiomeric excess is ee >98%. Distillation allows the ethyl (S)-3-aminobutanoate to be isolated in pure form. Yield: 5.4 g (95%); enantiomeric excess: >98%.

What is claimed is:

1. A process for enantioselectively preparing 3-azidocarboxylic acid derivatives of the general formula (III) 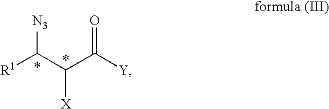
the process comprising:
reacting sulfonates of the general formula (II) 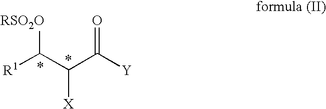
with an alkali metal azide, which comprises effecting the reaction in a solvent selected from the group consisting of carboxamides of the general formula (V) 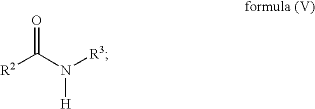
a solvent mixture comprising carboxamides of the general formula (V); a solvent mixture composed of water and a solvent miscible homogeneously with water; water; and DMSO;
wherein:
R¹ is a linear or branched, saturated or unsaturated alkyl, aryl or aralkyl radical which is cyclic or contains cyclic groups and is optionally substituted by Q, or is a carboxylate or carboxamide group;
Q is selected from the group consisting of carboxylato, carboxamido, halogen, cyano, nitro, acyl, silyl, silyloxy, aryl, heteroaryl, OR′, NR′R″ and SR′, where R′ and R″ are each independently hydrogen, a linear or branched, saturated or unsaturated alkyl, aryl or aralkyl radical which is cyclic or contains cyclic groups and is optionally substituted or contains a protecting group;
X is hydrogen or a radical as defined for R¹ or a substituent as defined for Q;
Y is a radical selected from the group comprising OR′, NR′R″ and SR′, where R′ and R″ are each independently hydrogen, a suitable protecting group or a linear or branched, saturated or unsaturated alkyl, aryl or aralkyl radical which is cyclic or contains cyclic groups and is optionally substituted by Q;
R¹ and X are optionally joined to one another and may form an at least 5-membered ring;
R is a linear or branched, saturated or unsaturated alkyl, aryl or aralkyl radical which is cyclic or contains cyclic groups and is optionally substituted by Q; and
R² and R³ are each independently hydrogen or a linear or branched, saturated or unsaturated alkyl, aryl or aralkyl radical which is cyclic or contains cyclic groups and is optionally substituted by Q;
with the proviso that the addition of a phase transfer catalyst is not used the case of reaction in water.

2. The process of claim 1, wherein R is an alkyl radical selected from the group consisting of methyl, ethyl, n-propyl, isopropyl and butyl.

3. The process of claim 1, wherein X is hydrogen (H).

4. The process of claim 1, wherein the reaction is performed with 1-2 equivalents of sodium azide based on the sulfonate of the general formula (II) used.

5. The process of claim 1, wherein the reaction is performed in a solvent mixture composed of water and a solvent which is miscible homogeneously with water and is selected from the group comprising dimethyl sulfoxide, dimethylformamide, formamide or N-methylformamide.

6. The process of claim 1, wherein the reaction is performed in formamide or N-methylformamide.

7. The process of claim 1, wherein the reaction is performed in DMSO.

8. The process of claim 1, wherein a resulting 3-azidocarboxylic acid derivatives are reduced in a subsequent step to 3-aminocarboxylic acid derivatives of the general formula (IV)

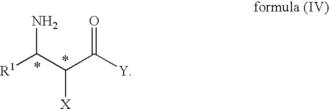

9. The process of claim 8, wherein the 3-azidocarboxylic acid derivatives or 3-aminocarboxylic acid derivatives are obtained in enantiomerically pure or enantiomerically enriched form.

10. The process of claim 9, wherein R¹ is a linear or branched, saturated or unsaturated alkyl or aralkyl radical which is cyclic or contains cyclic groups and wherein R¹ optionally substituted by Q.

11. The process of claim 9, wherein R¹ is selected from the group consisting of methyl, ethyl, propyl, isopropyl, n-butyl, isobutyl, tert-butyl, pentyl, hexyl, heptyl, octyl, nonyl and benzyl.

12. A process for enantioselectively preparing 3-azidocarboxylic acid derivatives of the general formula (III) 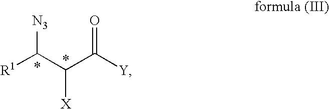
the process comprising:
reacting sulfonates of the general formula (II) 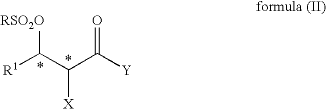
with an alkali metal azide, which comprises effecting the reaction in a solvent,
the solvent being selected from the group consisting of dimethyl sulfoxide; dimethylformamide; formamide; N-methylformamide; a solvent mixture which comprises dimethyl sulfoxide, dimethylformamide, formamide or N-methylformamide; a solvent mixture composed of water and dimethyl sulfoxide, dimethylformamide, formamide or N-methylformamide; and water
wherein:
R¹ is a linear or branched, saturated or unsaturated alkyl, aryl or aralkyl radical which is cyclic or contains cyclic groups and is optionally substituted by Q, or is a carboxylate or carboxamide group;
Q is selected from the group consisting of carboxylato, carboxamido, halogen, cyano, nitro, acyl, silyl, silyloxy, aryl, heteroaryl, OR′, NR′R″ and SR′, where R′ and R″ are each independently hydrogen, a linear or branched, saturated or unsaturated alkyl, aryl or aralkyl radical which is cyclic or contains cyclic groups and is optionally substituted or contains a protecting group;
X is hydrogen or a radical as defined for R¹ or a substituent as defined for Q;
Y is a radical selected from the group comprising OR′, NR′R″ and SR′, where R′ and R″ are each independently hydrogen, a suitable protecting group or a linear or branched, saturated or unsaturated alkyl, aryl or aralkyl radical which is cyclic or contains cyclic groups and is optionally substituted by Q;
R¹ and X are optionally joined to one another and may form an at least 5-membered ring;
R is a linear or branched, saturated or unsaturated alkyl, aryl or aralkyl radical which is cyclic or contains cyclic groups and is optionally substituted by Q; and
with the proviso that the addition of a phase transfer catalyst is not used the case of reaction in water.

13. The process of claim 12, wherein X is hydrogen (H).

14. The process of claim 12, wherein the reaction is performed with 1-2 equivalents of sodium azide based on the sulfonate of the general formula (II) used.

15. The process of claim 12, wherein the reaction is performed in formamide or N-methylformamide.

16. The process of claim 12, wherein a resulting 3-azidocarboxylic acid derivatives is reduced in a subsequent step to 3-aminocarboxylic acid derivatives of the general formula (IV)

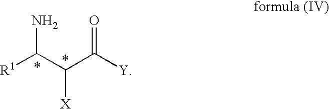

17. The process of claim 16, wherein the 3-azidocarboxylic acid derivatives or 3-aminocarboxylic acid derivatives are obtained in enantiomerically pure or enantiomerically enriched form.

18. The process of claim 16, wherein R¹ is a linear or branched, saturated or unsaturated alkyl or aralkyl radical which is cyclic or contains cyclic groups and wherein R¹ optionally substituted by Q.

19. The process of claim 16, wherein R¹ is selected from the group consisting of methyl, ethyl, propyl, isopropyl, n-butyl, isobutyl, tert-butyl, pentyl, hexyl, heptyl, octyl, nonyl and benzyl.

20. A process for enantioselectively preparing 3-azidocarboxylic acid derivatives of the general formula (III) 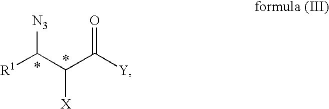
the process comprising:
a) reacting sulfonates of the general formula (II) 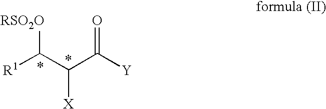
with an alkali metal azide to form an initial product, the reaction being effected in a solvent selected from the group consisting of carboxamides of the general formula (V) 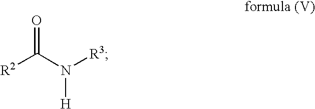
a solvent mixture comprising carboxamides of the general formula (V); a solvent mixture composed of water and a solvent miscible homogeneously with water; water; and DMSO; and
b) reducing the initial product to 3-aminocarboxylic acid derivatives of the general formula (IV) 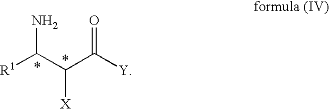
wherein:
R¹ is a linear or branched, saturated or unsaturated alkyl, aryl or aralkyl radical which is cyclic or contains cyclic groups and is optionally substituted by Q, or is a carboxylate or carboxamide group;
Q is selected from the group consisting of carboxylato, carboxamido, halogen, cyano, nitro, acyl, silyl, silyloxy, aryl, heteroaryl, OR′, NR′R″ and SR′, where R′ and R″ are each independently hydrogen, a linear or branched, saturated or unsaturated alkyl, aryl or aralkyl radical which is cyclic or contains cyclic groups and is optionally substituted or contains a protecting group;
X is hydrogen or a radical as defined for R¹ or a substituent as defined for Q;
Y is a radical selected from the group comprising OR′, NR′R″ and SR′, where R′ and R″ are each independently hydrogen, a suitable protecting group or a linear or branched, saturated or unsaturated alkyl, aryl or aralkyl radical which is cyclic or contains cyclic groups and is optionally substituted by Q;
R¹ and X are optionally joined to one another and may form an at least 5-membered ring;
R is a linear or branched, saturated or unsaturated alkyl, aryl or aralkyl radical which is cyclic or contains cyclic groups and is optionally substituted by Q; and
R² and R³ are each independently hydrogen or a linear or branched, saturated or unsaturated alkyl, aryl or aralkyl radical which is cyclic or contains cyclic groups and is optionally substituted by Q;
with the proviso that the addition of a phase transfer catalyst is not used the case of reaction in water.
